(19) United States
(12) Patent Application Publication
(10) Pub. No.: US 2009/0260997 A1
(43) Pub. Date: Oct. 22, 2009

(54) Single Use Packaging For Dental Impression Material

(75) Inventor: Frank Rovelli, Ventura, CA (US)

Correspondence Address: KOPPEL, PATRICK, HEYBL & DAWSON, 2815 Townsgate Road, SUITE 215, Westlake Village, CA, 91361-5827, US

(73) Assignee: Dux Industries, Inc.

(21) Appl. No.: 12/104,332

(22) Filed: Apr. 16, 2008

(51) Int. Cl. A61B 19/02

(52) U.S. Cl. 206/63.5

(57) ABSTRACT

A package for containing a preset quantity of a dry powder material is marked on its exterior with instructions for opening the package for transfer of its contents to a mixing container and cutting instructions, which may be the same, such that when the cutting instructions are followed a measuring container is created which has an internal volume preset to hold no more than the required quantity of liquid to be mixed with the dry powder material to create a wet mixture with desired handling properties.

The present invention relates to packaging for a powdered material to be subsequently mixed with a liquid in a predefined proportion to produce a solution or paste of the combined powder and liquid. More particularly, the packaging is intended as a container for a fixed quantity of a powdered material which must be blended with a defined quantity of water to form an impression material with a preset consistency for dental use.

### BACKGROUND

Dental alginate impression material, dental plaster, dental filling and restoration material and dental cement are used in various different dental procedures. These materials are generally provided in bulk packaging as a powder. The term alginate impression material, which is typically referred to in the dental field as just “alginate”, refers to an irreversible alginate-based hydrocolloid. The alginate component of the impression material comprises a salt of alginic acid (a colloidal substance from brown seaweed) used, in the form of calcium, sodium or ammonium alginate. This salt is generally combined with sodium or calcium sulfate dehydrate, phosphates or carbonates and a reinforcing filler such as diatomaceous earth. The amount of powdered alginate impression material and water required varies depending upon the brand used and the consistency and setting time desired for the mixed impression material. The quantity of water to be added is provided by the impression material manufacturer but generally ranges on a weight to weight basis (the density of water is approximately 1.0 gm/ml) from about 1.4 ml to about 3.1 ml of water or aqueous solution (may include flavoring agents colorants or other liquid additives) to 1 gm of dry powdered alginate impression material.

A desired quantity of the powdery material for the procedure is typically transferred to a mixing vessel and a specific quantity of water or an aqueous solution, the quality of water depending on the quantity of powdered material and the chemical characteristics of that material being used, must then be measured out and added to the mixing container. If the correct ratio of powder to water is used a malleable composition is formed. When it is desired to take patient's tooth impression using an alginate impression material, the quantity of the impression material powder required for the clinical procedure is conventionally measured with a measuring cup, and then the measured quantity of powder and a proper amount of water are mixed and kneaded to a desired state suitable for the impression taking or impression molding. Depending on the size of the impression to be taken, a different quantity of the alginate impression material may be used. For example, for a full jaw 21 gms might be used, a half jaw might use 14 grams and 7 grams might be used for 2-3 teeth.

U.S. Pat. No. 4,689,079 is an example of single dose packaging of alginate and discloses a single unit package for 2-3 teeth. For larger impressions, two packages would be used for a half mouth or three packages for a full mouth impression. Alternatively, three different sized unit packages might be provided. The contents of the package are placed in a mixing bowl, the required quantity of water is measured using a measuring cup and added to the mixing bowl, and the components are stirred until completely blended.

Instead of using a mixing bowl, a pouch, such as shown in U.S. Pat. No. 5,709,467, may be provided to receive both the measured quantity of powder and water. The pouch is then sealed and the contents mixed within the pouch until the proper consistency is obtained.

Another approach is a unit dose package containing the dry, powdered impression material. The package also has adequate space therein to receive the proper quantity of water which is added to the pouch using a syringe. The contents of the pouch are then mixed within the original package until the proper consistency is obtained. (See U.S. Pat. Nos. 5,052,554 and 5,465,833).

A further unit dose packaging concept for impression material is to provide in a single divided pouch a unit of dry powdered material in a first compartment and, separated therefrom in a second compartment, the required quantity of liquid. The barrier between the compartments is removed or broken and the contents mixed within the original package until the proper consistency is obtained. U.S. Pat. No. 4,023,675 show a tube with the components separated by a clamped central portion.

### SUMMARY

A pouch contains an appropriate amount of a powdered component for use to prepare a wet impression material, the volume thereof being adequate for holding sufficient powdered material for a single impression tray. The pouch has markings on its outer surface for the user to follow so that once the container is cut along the markings and the contents are removed a designated portion of that pouch is a proper sized measuring cup for a preset quantity of water or other liquids necessary to be added to the powdered material to produce the optimum final composition.

### DESCRIPTION OF DRAWINGS

FIG. 1 is a front perspective view of a package incorporating features of the invention.

FIG. 2 is side view of a first embodiment of the package of claim 1 cut along line 2-2.

FIG. 3 is side view of a second embodiment of the package of claim 1 cut along line 2-2.

FIG. 4 is a front view of the package of claim 1,

FIG. 5 is a front view of a further embodiment of the package of FIG. 1.

FIG. 6 is a front view of a still further embodiment of the package FIG. 1.

### DETAILED DESCRIPTION

Typically, the powdered impression material is provided in a bulk package and the dentist or dental technician must remove a measured quantity of the powdered material from the bulk package, determine from the suppliers literature or the instruction on the package the appropriate quantity of water to be mixed with the powdered impression material and measure and mix the water with the powdered material. Because the dentist may use impression material from different suppliers, or different impression materials from the same suppliers this ratio of liquid to powder may be different each time a batch of impression material is prepared.

In accordance with the present invention the powdered alginate impression material is provided in a single procedure package or pouch 10 that contains a quantity of powdered material 12 necessary for preparing sufficient wet impression material for a single impression tray. A typical pouch would be formed from a moisture and water impermeable film such as an aluminum or other metal film formed into a tube, cut into a suitable length and sealed at both ends with the powdered contents therein. The metal film may also have a meltable polymer film on the inner surface thereof so the ends can be heat sealed, an adhesive material can be applied to the ends being sealed, or the ends can be sealed by folding and crimping the ends of the metallic foil tube. In a further embodiment, the pouch can be an extruded plastic tube which can be heat or adhesively sealed such as used in form, fill and seal techniques common in the packaging industry. Access to the contents of the package is obtained by cutting along a cut line 14 printed on the package or a label attached to the pouch 10. Once the powdered material 12 is removed from the single procedure package 10 a designated portion created by cutting the package along the cut line 14 on the package is then used as the measuring vessel for the water. It is important to recognize that, while the ratio of water to powder material on a weight basis is generally greater than 1:1 and typically from about 1.5:1 to as much as 2.8:1, the volume of water to volume of powdered material in the mix is generally less than 1:1.

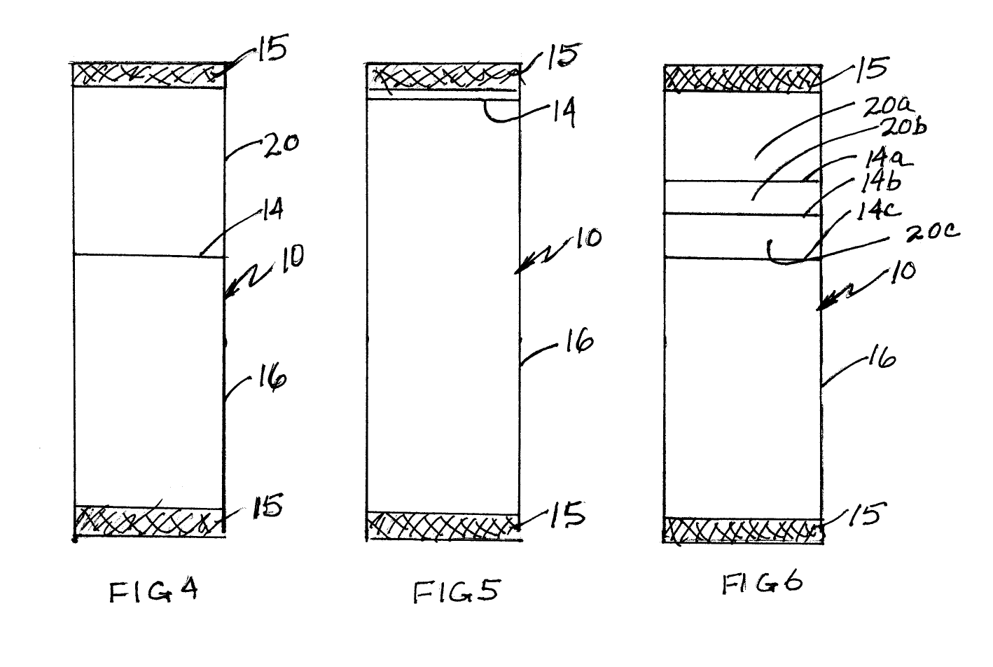

Referring to FIGS. 1-5, a pouch 10 incorporating features of the invention is shown. The pouch 10 is typically formed from plastic coated aluminum foil with first and second heat sealed end portions 15, the second seal being applied after the pouch receives the desired quantity of powdered material 12. The dimensions of the pouch 10 are chosen so that when the pouch is held in a vertical orientation so that the powdered impression material is in a lower portion 16 of the pouch 10 there is a void space 18 above the powdered material comprising a second portion 20 that has an inner volume corresponding with the preferred amount of water necessary for making the proper wet mixture. A cut line 14 is printed on the pouch at a position above the top of the powdered material 12 therein but at a location on the pouch 10 such that when the user opens the pouch by cutting along the cut line 14 the upper portion 20 of the package 10 has an internal volume, specified by the manufacturer, for holding the quantity of water appropriate for the powdered contents 12 which was contained in that package 10. Once the user has poured the powdered contents 12 from the lower or first portion 16 of the package 10 into a mixing vessel, that first portion 16 is discarded and the upper, second portion 20 is used to measure the quantity of water required for the wet composition.

In a typical single dose (full jaw) package, such as shown in FIG. 1, the pouch contains 22 grams of powdered alginate material. By cutting along the cut line 14 an upper portion 20 is created that can contain, when filled to the cut line 14, 48 ml of water. Mixing the powdered alginate with the 48 ml of water results in 59 ml of wet impression material.

Listed in Table 1 are six additional representatives commercial product mixing ratios.

TABLE 1

| Product | Water (ml) | Powder (gm) | Water to Powder Ratio |
|---|---|---|---|
| A | 16.0 | 6.0 | 2.7 |
| B | 19.0 | 7.0 | 2.7 |
| C | 25.0 | 8.0 | 3.1 |
| D | 16.0 | 6.7 | 2.4 |
| E | 13.3 | 6.0 | 2.2 |
| F | 18.0 | 9.0 | 2.0 |

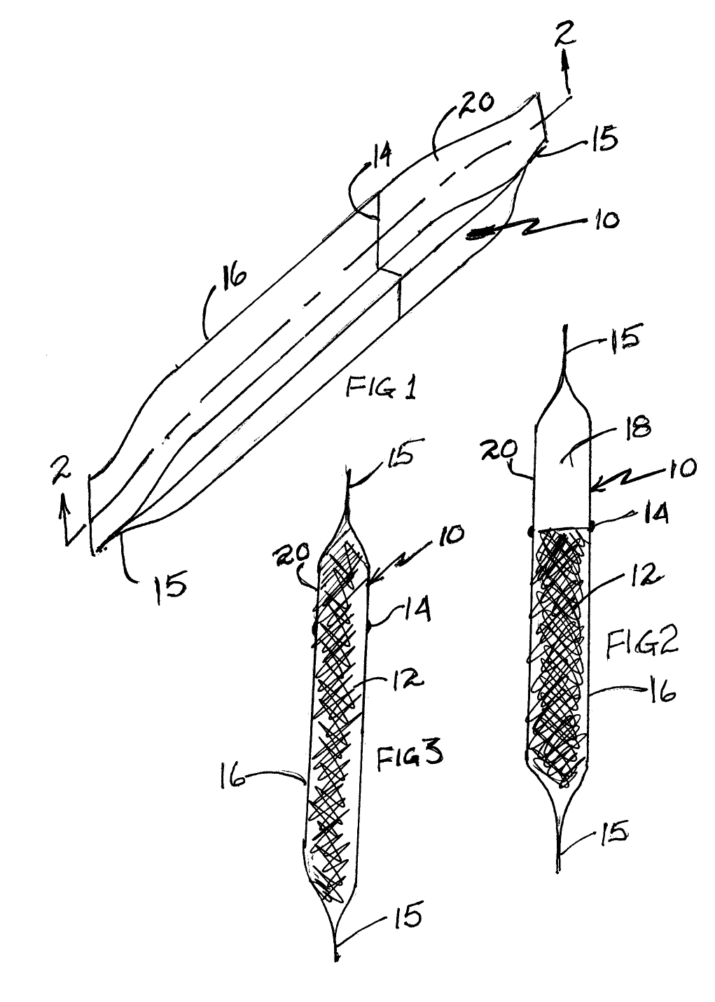

This pouch 10 can be configured in different arrangements. In the first arrangement shown in FIG. 2, when the pouch 10 is held in a vertical position there is sufficient internal volume to allow all of the powdered alginate to reside below the cut line 14 and the upper portion 20 which is above the cut line has a void space 18 equal to the desired water volume. This allows the package 10 to be cut as directed without fear of loosing some of the powdered alginate. In the alternative, the internal volume of the pouch 10 can be filled with powdered alginate such as shown in FIG. 3. Cutting along the cut line 14 still results in the creation of a water measuring portion. However, because the pouch is filled with powdered material 12, during opening it must be held in a horizontal orientation over the mixing bowl (not shown) as the powdered contents will start to fall from the pouch 10 as soon as the cut is started. Either of these designs can be used when the volume of water is less than the dry volume of the alginate.

FIGS. 3-5 show the external appearance of three different pouches 10 intended to handle different liquid to powder volume ratios. It is important to point out that volume ratios and weight ratios are different as the powdered alginate material is more voluminous than the same material compacted or when wetted. FIG. 3 illustrates a pouch 10 where the volume ratio of water to powder is less than 1:1 or greater than 1:1. Where the water to powder volume ratio is less than 1:1 the cutline 14 is positioned so the volume of the second portion 20 of the pouch 10 can be used as a measuring cup for the appropriate quantity of water. In the alternative, if the water to powder volume ratio is greater than 1:1, the cutline 14 is positioned so that the bottom or first portion 16 of the pouch 10 can be used as the measuring cup for the water.

Referring to FIG. 5, if the volume of water and volume of dry powdered alginate 12 are equal or substantially equal the cut line 14 can be at the top of the pouch 10 (adjacent the end seal 15) and the pouch portion previously holding the powder (the first portion 16), once emptied, now becomes the water measuring container. Alternatively, in the situation where the volume of water required is greater than the volume of dry powdered alginate the cut line 14 can be positioned near the end seal 15 of the pouch 10 such that the pouch volume holds the required quantity of water but the volume of powdered material 12 does not fill the pouch.

FIG. 6 illustrates a further variation wherein the pouch 10 showing three cut lines 14a, 14b, 14c so that the user can create three different second portions 20a, 20b, 20c respectively for receiving different volumes of water. This arrangement can be used where the powdered contents can be mixed with differing quantities of water to create different compositions with different properties such as viscosity or setting times. For example it might be desirable to produce a thicker, more viscose material for preparing impressions of the lower teeth as a lower tooth impression procedure requires the impression tray and material in it to be utilized with the impression material facing downward, which can result in the material flowing before it sets.

While the packaging arrangement has been described herein for use with powdered alginate materials, other powdered material, such as gypsum casting materials or cements for numerous different applications can be packaged using the unit packaging described herein.

One skilled in the art will recognize that there are numerous different applications outside of the dental field or the medical field where it is desirable to prepare a powder/water composition of a known quantity for a single use and it would be convenient to not require a separate measuring vessel be located and used. For example, numerous food products such as soups and salad dressings may be prepared in a powdered or dehydrated form requiring a fixed quantity of liquid to be added to the dry material. The single use package described herein could be suitable for such an application.

I claim:

1. A single use package for delivering a quantity of a dry material to a mixing vessel, said dry material to be mixed with a fixed amount of a liquid, to prepare a wet composition of preset properties for use in a single procedure comprising:
a pouch formed from a moisture and liquid impermeable material, said pouch configured to hold a specific quantity of the dry material, wherein said specific quantity of dry material when mixed with a specific volume of a liquid forms a wet end product, said pouch having on an outer surface thereof an indicia for use as a guide in cutting the pouch into first and second portions, one of said first and second portions having a predetermined volume usable as a measuring cup for receiving and transferring the specific volume of the liquid to the mixing vessel for blending with the specific quantity of dry material.

2. The single use package of claim 1 wherein the dry material is a powdered alginate material used in preparing a batch of a moist material for use in obtaining a single dental impression, said liquid being water.

3. The single use package of claim 2 wherein the pouch contains a fixed quantity of powdered alginate material, the moist material for use in obtaining a single dental impression produced therefrom requires a preset fixed volume of water and the volume capacity of second portion of the pouch following cutting of the pouch along the indicia thereon holds no more than said preset fixed volume of water.

4. A single use package for preparing a dental impression material comprising a moisture and liquid impermeable film formed into a sealed pouch having an enclosed volume, said pouch having sealed within said enclosed volume a quantity of dry powdered alginate for preparation of a single tray of dental impression material, the enclosed volume dividable into first and second portions by cutting the pouch along an indicia on the surface thereof, the volume of the first or second portions being selected to receive no more than a predetermine volume of water, the volume of water being at a predetermined ratio to the weight quantity of the dry powdered alginate.

5. The single use package of claim 4 containing 22 gms of powdered alginate and the first or second portion of the pouch having a volume of no more than 48 ml.

6. The single use package of claim 4 wherein the ratio of the volume of the powdered alginate to the first or second portions selected to receive the predetermine volume of water is greater than 1:1.

7. The single use package of claim 4 wherein the ratio of the volume of the powdered alginate to the first or second portions selected to receive the predetermine volume of water is from about 1.5:1 to about 3.1:1.

8. The single use package of claim 1 wherein the package is configured to hold from about 6 to about 9 gms of the dry material and one of the first or second portions is configured to hold from about 13 to about 25 ml of water.

9. The single use package of claim 8 wherein the package is configured to hold:
6 gms of powder and the first or second portion is configured to hold no more than 13.3 or 16 ml of water, or
6.7 gm of powder and the first or second portion is configured to hold 16 ml of water, or
7.0 gm of powder and the first or second portion is configured to hold 19 ml of water, or
8.0 gm of powder and the first or second portion is configured to hold 25 ml of water, or
9.0 gm of powder and the first or second portion is configured to hold 18 ml of water.

10. The single use package of claim 4 wherein the package is configured to hold from about 6 to about 9 gms of the dry material and one of the first or second portions is configured to hold from about 13 to about 25 ml of water.

11. The single use package of claim 10 wherein the package is configured to hold:
6 gms of powder and the first or second portion is configured to hold no more than 13.3 or 16 ml of water, or
6.7 gm of powder and the first or second portion is configured to hold 16 ml of water, or
7.0 gm of powder and the first or second portion is configured to hold 19 ml of water, or
8.0 gm of powder and the first or second portion is configured to hold 25 ml of water, or
9.0 gm of powder and the first or second portion is configured to hold 18 ml of water.

12. The single use package of claim 4 wherein the ratio of the volume of the powdered alginate to the volume of the first or second portions selected to receive the predetermine volume of water is from about 2.0:1 to about 3.1:1.
